(19) United States
(12) Patent Application Publication
(10) Pub. No.: US 2022/0254043 A1
(43) Pub. Date: Aug. 11, 2022

(54) A compression area identification platform and method thereof using content analysis

(71) Applicant: HU, Feiqing, Ningbo City, Zhejiang (CN)

(72) Inventor: Feiqing Hu, Ningbo City, Zhejiang (CN)

(21) Appl. No.: 17/428,571

(22) PCT Filed: Aug. 18, 2020
(86) PCT No.: PCT/CN2020/109758

§ 371 Date: Aug. 4, 2021

(30) Foreign Application Priority Data
Jul. 8, 2020 (CN) 202010648779.3

(51) Int. Cl. G06T 7/50; A61B 17/34; G06T 5/00; A61B 90/00

(52) U.S. Cl. CPC G06T 7/50; A61B 17/3403; G06T 2207/30088; A61B 90/361; A61B 90/37; G06T 5/009

(57) ABSTRACT

A compression area identification platform and method thereof using content analysis, wherein the platform includes: a compression identification device for acquiring each imaging depth of field value of each skin imaging pixel point in a skin imaging area, and calculating the mean value of each imaging depth of field value to obtain the reference depth of field; the compression identification device is also used in areas within the skin imaging area, and issuing compression area identification signal if the magnitude of imaging depth of field value greater than the reference depth of field exceeds a preset magnitude threshold in overrun of skin imaging pixel points, or otherwise, to issue the compression area unidentified signal. The present invention can quickly identify the skin compression area at a surgical puncture position and perform an on-site signal alarm.

### TECHNICAL FIELD

The present invention relates to the field of surgical monitoring, in particular to a compression area identification platform and method thereof using content analysis.

### BACKGROUND ART

Surgery refers to the excision, suture and other treatments performed by doctors on the patient's body with medical equipment. The operation of knives, scissors, needles and other equipment on the part of the human body to maintain the health of the patient. It is the main treatment method in surgical department, commonly known as “operation”. The purpose is to treat or diagnose diseases, such as removing diseased tissues, repairing damage, transplanting organs, and improving the function and shape of the body.

Early surgery was limited to simple manual methods to cut, and stitch on the body surface, such as abscess drainage, tumor resection, and traumatic sutures. Therefore, surgery is an operation that destroys the integrity of the tissue (incision) or restores (suturing) the tissue whose integrity has been damaged. With the development of surgical science, the field of surgery continues to expand, and it can be performed on any part of the human body. The applied instruments are also constantly updated, such as electric scalpels, microwave scalpels, ultrasonic scalpels and laser scalpels. Therefore, surgery also has a broader meaning.

### SUMMARY OF THE INVENTION

In order to solve the technical problems in the related fields, the present invention provides a compression area identification platform using content analysis, which can quickly identify the skin compression area at a surgical puncture position and perform an on-site signal alarm, thereby reminding medical staff to adjust the puncture equipment or change the puncture position, to avoid injury to the patient and ensure the normal operation of the surgery.

To this end, the present invention needs to possess at least the following key invention points:

(1) Perform a targeted analysis of the compression area on the puncture position of the patient's intubation basing on the imaging characteristics of the skin compression area, so that when there is a compression area, perform the corresponding signal alarm operation, thereby effectively avoiding secondary injury to the patient;

(2) In order to limit the detection range of the compressed area, the skin area is first detected to perform on-site search of the compression area in the skin area.

According to an aspect of the present invention, a compression area identification platform using content analysis is provided, and the platform includes:

a compression identification device, which is connected to a skin analysis device, and is used for acquiring each imaging depth of field value of each skin imaging pixel point in a skin imaging area, and calculating the mean value of each imaging depth of field value to obtain the reference depth of field;

the compression identification device is also used in areas within the skin imaging area, and issuing compression area identification signal if the magnitude of imaging depth of field value greater than the reference depth of field exceeds a preset magnitude threshold in overrun of skin imaging pixel points, or otherwise, to issue the compression area unidentified signal;

a high-definition capture mechanism, which is used to perform a high-definition capture action on the puncture position of the patient's intubation to obtain and output the corresponding intubation scene image;

an image rendering device, which is connected to the high-definition capture mechanism, and is used to perform image rendering processing on the received intubation scene image to obtain and output a corresponding current rendered image;

a gamma correction device, which is connected to the image rendering device, and is used to perform gamma correction processing on the received current rendered image to obtain and output a corresponding gamma correction image;

a content-enriching device, which is connected to the gamma correction device, and is used to perform bilinear interpolation processing on the received gamma correction image to obtain and output a corresponding content processing image;

a skin analysis device, which is connected to the content-enriching device, and is used to identify the skin imaging pixel points in the content processing image, and outputting the area occupied by each skin imaging pixel point in the content processing image as a skin imaging area;

wherein, in the skin analysis device, the red component value of the skin imaging pixel point falls within the red component range, the blue component value falls within the blue component range, and the green component value falls within the green component range, and the red component range, the blue component range and the green component range are used to define the yellow range presented by the skin imaging area.

According to another aspect of the present invention, a compression area identification method using content analysis is also provided, wherein the method includes using the above-mentioned compression area identification platform using content analysis to perform real-time monitoring of the skin compression area at the patient's puncture position by basing on the analysis result of the live signal.

The compression area identification platform and method thereof using content analysis of the present invention is reliable in monitoring and has a wide range of applications. As the skin compression area at a surgical puncture position can be quickly identified and an on-site signal alarm can be performed, medical personnel are reminded to adjust the puncture equipment or change the puncture position.

### DETAILED IMPLEMENTATION METHOD

The implementation of the compression area identification platform and method thereof using content analysis of the present invention will be described in detail below.

The skin is divided into two layers: the epidermis and the dermis. The epidermis is on the skin surface and can be further divided into two parts, the stratum corneum and the germinal layer. The keratinized cells form the stratum corneum, which becomes dander after being shed. The cells in the germinal layer continue to divide and can replenish the stratum corneum that has fallen off. There are melanocytes in the germinal layer, and the melanin produced can prevent ultraviolet rays from damaging internal tissues. The epidermis is a stratified flat epithelium, while the dermis is a dense connective tissue with a lot of elastic fibers and collagen fibers, so it is elastic and tough. The dermis is thicker than the epidermis and is rich in blood vessels and nerves. There is subcutaneous tissue under the skin, which is a loose connective tissue with a large number of fat cells. The skin also has many appendages such as hair, sweat glands, sebaceous glands, nails and so on.

During the surgery, if there is skin compression in the buried skin area of the infusion tube or the blood-suction tube, it is necessary to perform on-site tube correction in time to avoid long-term skin compression causing discomfort and harm to the patient. However, there is no relevant identification mechanism in the prior art.

In order to overcome the above-mentioned shortcomings, the present invention builds a platform and method for identifying the compression area using content analysis, which can effectively solve the corresponding technical problems.

According to the embodiment of the present invention, the compression area identification platform using content analysis includes:

a compression identification device, which is connected to a skin analysis device, and is used for acquiring each imaging depth of field value of each skin imaging pixel point in a skin imaging area, and calculating the mean value of each imaging depth of field value to obtain the reference depth of field;

the compression identification device is also used in areas within the skin imaging area, and issuing compression area identification signal if the magnitude of imaging depth of field value greater than the reference depth of field exceeds a preset magnitude threshold in overrun of skin imaging pixel points, or otherwise, to issue the compression area unidentified signal;

a high-definition capture mechanism, which is used to perform a high-definition capture action on the puncture position of the patient's intubation to obtain and output the corresponding intubation scene image;

an image rendering device, which is connected to the high-definition capture mechanism, and is used to perform image rendering processing on the received intubation scene image to obtain and output a corresponding current rendered image;

a gamma correction device, which is connected to the image rendering device, and is used to perform gamma correction processing on the received current rendered image to obtain and output a corresponding gamma correction image;

a content-enriching device, which is connected to the gamma correction device, and is used to perform bilinear interpolation processing on the received gamma correction image to obtain and output a corresponding content processing image;

a skin analysis device, which is connected to the content-enriching device, and is used to identify the skin imaging pixel points in the content processing image, and outputting the area occupied by each skin imaging pixel point in the content processing image as a skin imaging area;

wherein, in the skin analysis device, the red component value of the skin imaging pixel point falls within the red component range, the blue component value falls within the blue component range, and the green component value falls within the green component range, and the red component range, the blue component range and the green component range are used to define the yellow range presented by the skin imaging area.

Next, the specific structure of the compression area identification platform using content analysis of the present invention will be further illustrated.

In the compression area identification platform using content analysis:

the skin analysis device, the image rendering device, the gamma correction device, and the content-enriching device are all set in an instrument box near the hospital bed where the patient is located.

In the compression area identification platform using content analysis:

the skin analysis device is implemented by a programmable logic device, and the programmable logic device is designed by using VHDL.

In the compression area identification platform using content analysis:

the image rendering device is a DSP processing chip, and the DSP processing chip has a built-in timer and a ROM memory.

In the compression area identification platform using content analysis:

data connection and data interaction are performed between the skin analysis device and the image rendering device through a 16-bit parallel data interface.

In the compression area identification platform using content analysis:

the skin analysis device and the image rendering device share the same on-site timing device and the same power supply input device.

In the compression area identification platform using content analysis:

a data caching device is also provided between the skin analysis device and the image rendering device;

wherein, the data caching device is respectively connected with the skin analysis device and the image rendering device through two data interfaces.

The compression area identification platform using content analysis further includes:

a SRAM storage chip, which is arranged between the skin analysis device and the image rendering device, and is connected to the skin analysis device and the image rendering device respectively;

wherein, the SRAM storage chip is used to store current output data of the skin analysis device and the image rendering device respectively;

The compression area identification platform using content analysis further includes:

a GPRS communication interface, which is connected to the skin analysis device, and is used to send the current data sent by the skin analysis device through the GPRS communication line;

wherein, the SRAM storage chip is also used to store the red component range, the blue component range, and the green component range.

At the same time, in order to overcome the above-mentioned shortcomings, the present invention also builds a method for identifying the compression area using content analysis, wherein the method includes using the compression area identification platform using content analysis to perform real-time monitoring of the skin compression area at the patient's puncture position by basing on the analysis result of the live signal.

In addition, the DSP processing chip, also called a digital signal processor, is a microprocessor particularly suitable for digital signal processing operations, and its main application is to quickly implement various digital signal processing algorithms in real time.

According to the requirements of digital signal processing, DSP processing chips generally have the following main characteristics: (1) One multiplication and one addition can be completed in one instruction cycle; (2) Program and data space are separated, and instructions and data can be accessed at the same time; (3)) There is a fast RAM on-chip, which can usually be accessed in two blocks at the same time through independent data buses; (4) Hardware support with low or zero overhead loops and jumps; (5) Fast interrupt processing and hardware I/O support; (6) Multiple hardware address generators that operate in a single cycle; (7) Multiple operations can be executed simultaneously; (8) Pipeline operation is supported so that operations such as fetching, decoding and execution can be performed overlapped.

A person of ordinary skill in the art can understand that all or part of the steps carried in the method of the foregoing embodiments can be implemented by a program instructing relevant hardware to complete. The program can be stored in a computer-readable storage medium. When executed, it includes the steps of the method embodiment. The aforementioned storage media include: Read-Only Memory (abbreviated as: ROM), Random Access Memory (abbreviated as: RAM), magnetic disks or optical disks, etc., which can store program codes.

Finally, it should be noted that the above embodiments are only used to illustrate the technical solutions of the present invention, not to limit them; although the present invention has been described in detail with reference to the foregoing embodiments, those of ordinary skill in the art should understand that the technical solutions recorded in the foregoing embodiments can still be modified, or some or all of the technical features can be equivalently replaced; these modifications or replacements do not make the essence of the corresponding technical solutions deviate from the scope of technical solutions of the embodiments of the present invention.

1. A compression area identification platform using content analysis, wherein the platform includes:
a compression identification device, which is connected to a skin analysis device, and is used for acquiring each imaging depth of field value of each skin imaging pixel point in a skin imaging area, and calculating the mean value of each imaging depth of field value to obtain the reference depth of field;
the compression identification device is also used in areas within the skin imaging area, and issuing compression area identification signal if the magnitude of imaging depth of field value greater than the reference depth of field exceeds a preset magnitude threshold in overrun of skin imaging pixel points, or otherwise, to issue the compression area unidentified signal;
a high-definition capture mechanism, which is used to perform a high-definition capture action on the puncture position of the patient's intubation to obtain and output the corresponding intubation scene image;
an image rendering device, which is connected to the high-definition capture mechanism, and is used to perform image rendering processing on the received intubation scene image to obtain and output a corresponding current rendered image;
a gamma correction device, which is connected to the image rendering device, and is used to perform gamma correction processing on the received current rendered image to obtain and output a corresponding gamma correction image;
a content-enriching device, which is connected to the gamma correction device, and is used to perform bilinear interpolation processing on the received gamma correction image to obtain and output a corresponding content processing image;
a skin analysis device, which is connected to the content-enriching device, and is used to identify the skin imaging pixel points in the content processing image, and outputting the area occupied by each skin imaging pixel point in the content processing image as a skin imaging area;
wherein, in the skin analysis device, the red component value of the skin imaging pixel point falls within the red component range, the blue component value falls within the blue component range, and the green component value falls within the green component range, and the red component range, the blue component range and the green component range are used to define the yellow range presented by the skin imaging area.

2. The compression area identification platform using content analysis according to claim 1, characterized in that:
the skin analysis device, the image rendering device, the gamma correction device, and the content-enriching device are all set in an instrument box near the hospital bed where the patient is located.

3. The compression area identification platform using content analysis according to claim 2, characterized in that:
the skin analysis device is implemented by a programmable logic device, and the programmable logic device is designed by using VHDL.

4. The compression area identification platform using content analysis according to claim 3, characterized in that:
the image rendering device is a DSP processing chip, and the DSP processing chip has a built-in timer and a ROM memory.

5. The compression area identification platform using content analysis according to claim 4, characterized in that:
data connection and data interaction are performed between the skin analysis device and the image rendering device through a 16-bit parallel data interface.

6. The compression area identification platform using content analysis according to claim 5, characterized in that:
the skin analysis device and the image rendering device share the same on-site timing device and the same power supply input device.

7. The compression area identification platform using content analysis according to claim 6, characterized in that:
a data caching device is also provided between the skin analysis device and the image rendering device;
wherein, the data caching device is respectively connected with the skin analysis device and the image rendering device through two data interfaces.

8. The compression area identification platform using content analysis according to claim 7, characterized in that, the platform further includes:
a SRAM storage chip, which is arranged between the skin analysis device and the image rendering device, and is connected to the skin analysis device and the image rendering device respectively;
wherein, the SRAM storage chip is used to store current output data of the skin analysis device and the image rendering device respectively;

9. The compression area identification platform using content analysis according to claim 8, characterized in that, the platform further includes:
a GPRS communication interface, which is connected to the skin analysis device, and is used to send the current data sent by the skin analysis device through the GPRS communication line;
wherein, the SRAM storage chip is also used to store the red component range, the blue component range, and the green component range.

10. The compression area identification method using content analysis, wherein the method includes using the compression area identification platform using content analysis according to any of claim 1 to perform real-time monitoring of the skin compression area at the patient's puncture position by basing on the analysis result of the live signal.
